(19) United States
(12) Patent Application Publication
(10) Pub. No.: US 2014/0217649 A1
(43) Pub. Date: Aug. 7, 2014

(54) TISSUE ENGINEERED CONSTRUCTS

(71) Applicant: The General Hospital Corporation, Boston, MA (US)

(72) Inventors: Irene E. Kochevar, Charlestown, MA (US); Robert W. Redmond, Brookline, MA (US); Anne C. O'Neill, Dublin (IE); Mark Randolph, Chelmsford, MA (US)

(73) Assignee: The General Hospital Corporation, Boston, MA (US)

(21) Appl. No.: 14/152,439

(22) Filed: Jan. 10, 2014

Related U.S. Application Data
(63) Continuation of application No. 12/991,643, filed on Feb. 24, 2011, now abandoned.
(60) Provisional application No. 61/052,160, filed on May 9, 2008.

(51) Int. Cl. A61B 17/11

(52) U.S. Cl. CPC A61B 17/1128
USPC 264/494

(57) ABSTRACT

The present invention relates to a field of biocompatible membranes, tubes and conduits which comprising a photosensitizer which is capable of being crosslinked to form a three dimensional structure which can be implanted into a subject to assist in tissue bonding and nerve maintenance and development. Methods of making such membranes, tubes and conduits and kits comprising them are also described.

### RELATED APPLICATIONS

This application is a continuation of U.S. application Ser. No. 12/991,643, which is the U.S. National Phase pursuant to 37 U.S.C. §371 of International application No. PCT/US2009/043340, filed May 8, 2009, designating the United States and published in English on Nov. 12, 2009 as publication No. WO 2009/137793, which claims priority to U.S. provisional application Ser. No. 61/052,160, filed May 9, 2008. The entire disclosures of each of the foregoing patent applications are incorporated herein by reference.

### GOVERNMENT SUPPORT

Research supporting this application was supported by the DOD Medical Free Electron Laser Program. The government has certain rights in the invention.

### FIELD OF THE INVENTION

The present invention relates to a field of biocompatible membranes, tubes and conduits which comprising a photosensitizer which is capable of being crosslinked to form a three dimensional structure which can be implanted into a subject to assist in tissue bonding and nerve maintenance and development.

### BACKGROUND OF THE INVENTION

Surgical management of the nerve gap remains a significant challenge for the reconstructive surgeon. The current standard of care requires the harvest of nerve grafts for interposition between the nerve ends, resulting in an inevitable neurological deficit at the donor site. Recent research has focused on the development of alternative methods of bridging the nerve gap. Biocompatible nerve guidance conduits have been developed using a number of biological and engineered materials in an attempt to avoid the need for autologous tissue.

Photochemical tissue bonding (PTB) is a promising new tissue repair technique. Visible laser light is combined with a photoreactive dye to create chemical bonds between the tissue surfaces. This technique has been successfully applied in a number of experimental tissue repair models. It has been previously demonstrated that PTB can be effectively used for peripheral nerve repair (Johnson et al 2006, in press). This work indicated that circumferential bonding at the repair site resulted in excellent preservation of neural architecture. It has also been shown that photochemical sealing of the repair site can enhance the histological and functional outcome of peripheral neurorrhaphy.

To permit neural regeneration, guidance tubes must have sufficient mechanical strength to resist collapse in-vivo. Conventional cross-linking techniques include chemical cross-linking using glutaraldehyde, formaldehyde or polyepoxy compounds and physical cross-linking using gamma irradiation, ultraviolet irradiation or heat treatments. A major disadvantage of these techniques is the time required to achieve sufficient cross-linking, which may be hours or even days.

Accordingly, there remains a need for a rapidly cross-linked nerve conduit and methods for making such conduits which can optimize the local environment for regeneration across the nerve gap with minimal toxicity and which are easier to fabricate and implant.

### SUMMARY OF THE INVENTION

In one aspect, the invention provides a tissue sealing device comprising a shaped biocompatible material, said material comprising at least a first section of cross-linked moieties and at least a second section of uncross-linked moieties, wherein said first and second sections are configured so that said second section is contactable with a tissue to be sealed and wherein said uncross-linked moieties can be cross-linked with proteins of said tissue to be sealed upon contact of said second section and said tissue with a photosensitizer agent and irradiation with electromagnetic energy.

In certain aspects, the photosensitizer agent of a tissue sealing device of the invention is selected from the group consisting of xanthene (including, but not limited to Rose Bengal), flavin, phenothiazine, triphenylmethyl, cyanine, Mono azo dye, Azine mono azo dye, Phenothia-zine dye, rhodamine dye, Benzyphen-oxazine dye, oxazine, anthroqui-none dye, and porphyrin.

In other aspects, the cross-linked moieties of a tissue sealing device of the invention are proteins.

In still other aspects, the biocompatible material of a tissue sealing device of the invention is a biocompatible membrane, including, but not limited to amniotic membrane (including, but not limited to human amniotic membrane), SIS, fascia, dura matter, peritoneum, and pericardium.

In some aspects of a tissue sealing device of the invention, the biocompatible material is in the shape of a tube.

In certain aspects, the second section of a tissue sealing device of the invention is a border region. In certain aspects, particularly when the biocompatible material of the tissue sealing device of the invention is in the shape of a tube, the border region can be at one or both ends of said material.

In yet other aspects, a tissue sealing device of the invention is cross-linked with electromagnetic energy applied at an irradiance less than 1.5 W/cm²², in some cases of about 0.50 W/cm².

In another aspect, the invention provides a tissue sealing device preform comprising a biocompatible material having at least a first section and a second section, wherein said first section includes a photosensitizer agent and said second section is free of said photosensitizer agent, such that when said preform is irradiated with electromagnetic energy, moieties in said first section are crosslinked to other moieties of said material and moieties in said second section remain uncrosslinked.

In some aspects, the cross-linked moieties of a tissue sealing preform of the invention are proteins.

In certain aspects, the photosensitizer agent of a tissue sealing preform of the invention is selected from the group consisting of xanthene (including, but not limited to Rose Bengal), flavin, phenothiazine, triphenylmethyl, cyanine, Mono azo dye, Azine mono azo dye, Phenothia-zine dye, rhodamine dye, Benzyphen-oxazine dye, oxazine, anthroquinone dye, and porphyrin.

In still other aspects, the biocompatible material of a tissue sealing preform of the invention is a biocompatible membrane, including, but not limited to amniotic membrane (including, but not limited to human amniotic membrane), SIS, fascia, dura matter, peritoneum, and pericardium.

In some aspects of a tissue sealing preform of the invention, the biocompatible material is in the shape of a tube.

In certain aspects, the second section of a tissue sealing preform of the invention is a border region. In certain aspects, particularly when the biocompatible material of the tissue sealing device of the invention is in the shape of a tube, the border region can be at one or both ends of said material.

In yet other aspects, a tissue sealing preform of the invention is cross-linked with electromagnetic energy applied at an irradiance less than 1.5 W/cm², in some cases of about 0.50 W/cm².

In another aspect, the invention provides a three-dimensional biocompatible structure comprising a biocompatible material in the shape of said structure, said structure comprising least a first section of cross-linked moieties and at least a second section of uncross-linked moieties, wherein said first and second sections are configured so that said second section is contactable with a tissue and wherein said uncross-linked moieties can be cross-linked with proteins of said tissue upon contact of said second region and said tissue with a photosensitizer agent and irradiation with electromagnetic energy.

In certain aspects, the biocompatible material of a three-dimensional biocompatible structure of the invention is a biocompatible membrane, including, but not limited to amniotic membrane (including, but not limited to human amniotic membrane), SIS, fascia, dura matter, peritoneum, and pericardium.

In other aspects, the photosensitizer agent of a three-dimensional biocompatible structure of the invention is selected from the group consisting of xanthene (including, but not limited to Rose Bengal), flavin, phenothiazine, triphenylmethyl, cyanine, Mono azo dye, Azine mono azo dye, Phenothia-zine dye, rhodamine dye, Benzyphen-oxazine dye, oxazine, anthroqui-none dye, and porphyrin.

In still other aspects of a three-dimensional biocompatible structure of the invention, the biocompatible material is in the shape of a tube.

In certain aspects, the second section of a three-dimensional biocompatible structure of the invention is a border region. In certain aspects, particularly when the biocompatible material of the tissue sealing device of the invention is in the shape of a tube, the border region can be at one or both ends of said material.

In yet other aspects, a three-dimensional biocompatible structure of the invention is cross-linked with electromagnetic energy applied at an irradiance less than 1.5 W/cm², in some cases of about 0.50 W/cm².

In another aspect, the invention provides a biocompatible conduit comprising a biocompatible material, said material comprising at least a first section of cross-linked moieties and at least a second section of uncross-linked moieties, wherein said first and second sections are configured so that said second section is contactable with a tissue and wherein said uncross-linked moieties can be cross-linked with proteins of said tissue upon contact of said second region and said tissue with a photosensitizer agent and irradiation with electromagnetic energy.

In some aspects, the cross-linked moieties of a biocompatible conduit of the invention are proteins.

In certain aspects, the biocompatible material of a biocompatible conduit of the invention is a biocompatible membrane, including, but not limited to amniotic membrane (including, but not limited to human amniotic membrane), SIS, fascia, dura matter, peritoneum, and pericardium.

In other aspects, the photosensitizer agent of a biocompatible conduit of the invention is selected from the group consisting of xanthene (including, but not limited to Rose Bengal), flavin, phenothiazine, triphenylmethyl, cyanine, Mono azo dye, Azine mono azo dye, Phenothia-zine dye, rhodamine dye, Benzyphen-oxazine dye, oxazine, anthroqui-none dye, and porphyrin.

In still other aspects of a biocompatible conduit of the invention, the biocompatible material or conduit is in the shape of a tube.

In certain aspects, the second section of a biocompatible conduit of the invention is a border region. In certain aspects, particularly when the biocompatible material of the tissue sealing device of the invention is in the shape of a tube, the border region can be at one or both ends of said material.

In yet other aspects, a biocompatible conduit of the invention is cross-linked with electromagnetic energy applied at an irradiance less than 1.5 W/cm², in some cases of about 0.50 W/cm².

In another aspect, the invention provides a biocompatible conduit comprising an amniotic membrane comprising at least a first section of cross-linked proteins and at least a second section of uncross-linked proteins, wherein said first and second sections are configured so that said second section is contactable with a tissue and wherein said uncrosslinked proteins can be cross-linked with proteins of said tissue upon contact of said second region and said tissue with a photosensitizer agent and irradiation with electromagnetic energy.

In some aspects, the photosensitizer agent of a biocompatible conduit of the invention is selected from the group consisting of xanthene (including, but not limited to Rose Bengal), flavin, phenothiazine, triphenylmethyl, cyanine, Mono azo dye, Azine mono azo dye, Phenothia-zine dye, rhodamine dye, Benzyphen-oxazine dye, oxazine, anthroquinone dye, and porphyrin.

In still other aspects of a biocompatible conduit of the invention, the biocompatible material is in the shape of a tube.

In certain aspects, the second section of a biocompatible conduit of the invention is a border region. In certain aspects, particularly when the biocompatible material of the tissue sealing device of the invention is in the shape of a tube, the border region can be at one or both ends of said material.

In yet other aspects, a biocompatible conduit of the invention is cross-linked with electromagnetic energy applied at an irradiance less than 1.5 W/cm², in some cases of about 0.50 W/cm².

In another aspect, the invention provides, a method of forming a shaped tissue sealing device, said method comprising: contacting at least a first section of a biocompatible material with a photosensitizer agent, wherein at least a second section of said biocompatible membrane is not contacted with said photosensitizer agent; forming said biocompatible material into a desired shape; applying electromagnetic energy to said biocompatible material in an amount and duration sufficient to form cross-links between moieties of said first section, whereby a shaped tissue sealing device is formed.

In certain aspects, the cross-linked moieties of method of forming a shaped tissue sealing device of the invention are proteins.

In certain aspects, the biocompatible material of the method of forming a shaped tissue sealing device of the invention is a biocompatible membrane, including, but not limited to amniotic membrane (including, but not limited to human amniotic membrane), SIS, fascia, dura matter, peritoneum, and pericardium.

In some aspects, the second section of a method of forming a shaped tissue sealing device of the invention is a border region.

In other aspects of the method of forming a shaped tissue sealing device, said shaped tissue sealing device has a three-dimensional shape, which may be a tube.

In still other aspects of the method of forming a shaped tissue sealing device, the photosensitizer agent is selected from the group consisting of xanthene (including, but not limited to Rose Bengal), flavin, phenothiazine, triphenylmethyl, cyanine, Mono azo dye, Azine mono azo dye, Phenothia-zine dye, rhodamine dye, Benzyphen-oxazine dye, oxazine, anthroqui-none dye, and porphyrin.

In yet other aspects of the method of forming a shaped tissue sealing device, the electromagnetic energy is applied at an irradiance less than 1.5 W/cm², in some cases of about 0.50 W/cm². In certain aspects of the method of forming a shaped tissue sealing device said electromagnetic energy is not applied to said second section.

In still yet another aspect, the method of forming a shaped tissue sealing device further comprises the step of obtaining said cross-linkable material.

In another aspect, the invention provides a method for making a biocompatible conduit, said method comprising: contacting at least a first section of a biocompatible material with a photosensitizer agent, wherein at least a second section of said biocompatible membrane is not contacted with said photosensitizer agent; forming said biocompatible material into a conduit; applying electromagnetic energy to said biocompatible material in an amount and duration sufficient to form cross-links between moieties of said first section, whereby a biocompatible conduit is formed.

In certain aspects, the cross-linked moieties of the method for making a biocompatible conduit of the invention are proteins.

In certain aspects, the biocompatible material of the method for making a biocompatible conduit of the invention is a biocompatible membrane, including, but not limited to amniotic membrane (including, but not limited to human amniotic membrane), SIS, fascia, dura matter, peritoneum, and pericardium.

In some aspects, the second section of the method for making a biocompatible conduit of the invention is a border region.

In still other aspects of the method for making a biocompatible conduit, the photosensitizer agent is selected from the group consisting of xanthene (including, but not limited to Rose Bengal), flavin, phenothiazine, triphenylmethyl, cyanine, Mono azo dye, Azine mono azo dye, Phenothia-zine dye, rhodamine dye, Benzyphen-oxazine dye, oxazine, anthroqui-none dye, and porphyrin.

In yet other aspects of the method for making a biocompatible conduit, the electromagnetic energy is applied at an irradiance less than 1.5 W/cm², in some cases of about 0.50 W/cm². In certain aspects of the method of forming a shaped tissue sealing device said electromagnetic energy is not applied to said second section.

In still yet another aspect, the method for making a biocompatible conduit further comprises the step of obtaining said cross-linkable material.

In another aspect, the invention provides a method for adhering neural tissue, comprising: contacting a neural tissue with a conduit, said conduit comprising a biocompatible material, said material comprising at least a first section of cross-linked moieties and at least a second section of uncross-linked moieties, wherein said neural tissue is contacted with the second section of the material; treating the neural tissue and/or the second section of the biocompatible material with a photosensitizing agent; and applying electromagnetic energy to the neural tissue and the second section of the biocompatible material in an amount and duration sufficient to form cross-links between proteins in the neural tissue and moieties the second section of the biocompatible material, thereby creating a tissue seal between the neural tissue and the conduit.

In some aspects of the method for adhering neural tissue, the photosensitizer agent is selected from the group consisting of xanthene (including, but not limited to Rose Bengal), flavin, phenothiazine, triphenylmethyl, cyanine, Mono azo dye, Azine mono azo dye, Phenothia-zine dye, rhodamine dye, Benzyphen-oxazine dye, oxazine, anthroqui-none dye, and porphyrin.

In other aspects of the method for adhering neural tissue, a circumferential, watertight seal is created between the neural tissues and the conduit.

In still other aspects of the method for adhering neural tissue, the intraneural neurotrophic environment is maintained within the conduit.

In certain aspects, the biocompatible material of the method for adhering neural tissue is selected from the group consisting of a blood vessel, acellular muscle and nerve. In other aspects, the biocompatible material of the method for adhering neural tissue is a synthetic absorbable polymer (including, but not limited to PGA). In still other aspects, the biocompatible material of the method for adhering neural tissue is human amniotic membrane.

In certain aspects, the cross-linked moieties of the method for adhering neural tissue of the invention are proteins.

In yet other aspects of the method for adhering neural tissue, the electromagnetic energy is applied at an irradiance less than 1.5 W/cm², in some cases of about 0.50 W/cm².

In another aspect, the method for adhering neural tissue, further comprises the step of forming said conduit. In still another aspect, in the method for adhering neural tissue, said step of contacting comprises placing said neural tissue inside said conduit.

In another aspect, the invention provides a method for adhering neural tissue, comprising: contacting a neural tissue with a conduit, said conduit comprising amniotic membrane, said amniotic membrane comprising at least a first section of cross-linked protein and at least a second section of uncross-linked protein, wherein said neural tissue is contacted with the second section of the amniotic membrane; treating the neural tissue and the second section of the amniotic membrane with a photosensitizing agent; and applying electromagnetic energy to the neural tissue and the second section of the amniotic membrane in an amount and duration sufficient to form cross-links between proteins in the neural tissue and moieties the second section of the amniotic membrane, thereby creating a tissue seal between the neural tissue and the conduit.

In some aspects of the method for adhering neural tissue, the photosensitizer agent is selected from the group consisting of xanthene (including, but not limited to Rose Bengal), flavin, phenothiazine, triphenylmethyl, cyanine, Mono azo dye, Azine mono azo dye, Phenothia-zine dye, rhodamine dye, Benzyphen-oxazine dye, oxazine, anthroqui-none dye, and porphyrin.

In other aspects of the method for adhering neural tissue, a circumferential, watertight seal is created between the neural tissues and the conduit.

In still other aspects of the method for adhering neural tissue, the intraneural neurotrophic environment is maintained within the conduit.

In yet other aspects of the method for adhering neural tissue, the electromagnetic energy is applied at an irradiance less than 1.5 W/cm², in some cases of about 0.50 W/cm².

In another aspect, the method for adhering neural tissue, further comprises the step of forming said conduit. In still another aspect, in the method for adhering neural tissue, said step of contacting comprises placing said neural tissue inside said conduit.

In another aspect, the invention provides a tissue sealing device comprising a shaped biocompatible material, said material comprising at least a first section of cross-linked moieties and at least a second section of uncross-linked moieties, wherein said first and second sections are configured so that said second section is contactable with a tissue to be sealed and wherein said uncross-linked moieties can be cross-linked with proteins of said tissue to be sealed upon contact of said second region and said tissue with a photosensitizer agent and irradiation with electromagnetic energy, said tissue sealing device produced by contacting said first section of said biocompatible material with a photosensitizer agent, wherein said second section of said biocompatible material is not contacted with said photosensitizer agent; forming said biocompatible material into a desired shape; applying electromagnetic energy to said biocompatible material wherein cross-links are formed between moieties of said first section, whereby a shaped tissue sealing device is formed.

In some aspects, the cross-linked moieties of a tissue sealing device of the invention are proteins.

In certain aspects, the photosensitizer agent of a tissue sealing device of the invention is selected from the group consisting of xanthene (including, but not limited to Rose Bengal), flavin, phenothiazine, triphenylmethyl, cyanine, Mono azo dye, Azine mono azo dye, Phenothia-zine dye, rhodamine dye, Benzyphen-oxazine dye, oxazine, anthroqui-none dye, and porphyrin.

In still other aspects, the biocompatible material of a tissue sealing device of the invention is a biocompatible membrane, including, but not limited to amniotic membrane (including, but not limited to human amniotic membrane), SIS, fascia, dura matter, peritoneum, and pericardium.

In some aspects of a tissue sealing device of the invention, the biocompatible material is in the shape of a tube.

In certain aspects, the second section of a tissue sealing device of the invention is a border region. In certain aspects, particularly when the biocompatible material of the tissue sealing device of the invention is in the shape of a tube, the border region can be at one or both ends of said material.

In yet other aspects, a tissue sealing device of the invention is cross-linked with electromagnetic energy applied at an irradiance less than 1.5 W/cm², in some cases of about 0.50 W/cm².

In another aspect, the invention provides a conduit comprising amniotic membrane, said membrane comprising at least a first section of cross-linked proteins and at least a second section of uncross-linked proteins, wherein said first and second sections are configured so that said second section is contactable with a tissue to be sealed and wherein said uncross-linked proteins can be cross-linked with proteins of said tissue to be sealed upon contact of said second region and said tissue with a photosensitizer agent and irradiation with electromagnetic energy, said conduit produced by contacting said first section of said amniotic membrane with a photosensitizer agent, wherein said second section of said amniotic membrane is not contacted with said photosensitizer agent; forming said amniotic membrane into a conduit; applying electromagnetic energy to said amniotic membrane wherein cross-links are formed between moieties of said first section, whereby a conduit is formed.

In certain aspects, the photosensitizer agent of a conduit of the invention is selected from the group consisting of xanthene (including, but not limited to Rose Bengal), flavin, phenothiazine, triphenylmethyl, cyanine, Mono azo dye, Azine mono azo dye, Phenothiazine dye, rhodamine dye, Benzyphen-oxazine dye, oxazine, anthroqui-none dye, and porphyrin.

In certain aspects, the second section of a conduit of the invention is a border region. In certain aspects—the border region can be at one or both ends of said material.

In yet other aspects, a conduit of the invention is cross-linked with electromagnetic energy applied at an irradiance less than 1.5 W/cm², in some cases of about 0.50 W/cm².

In another aspect, the invention provides a kit comprising the tissue sealing device of the invention, and packaging materials therefor.

In certain aspects, the photosensitizer agent of the kit is selected from the group consisting of xanthene (including, but not limited to Rose Bengal), flavin, phenothiazine, triphenylmethyl, cyanine, Mono azo dye, Azine mono azo dye, Phenothia-zine dye, rhodamine dye, Benzyphen-oxazine dye, oxazine, anthroqui-none dye, and porphyrin.

In other aspects, the cross-linked moieties of the kit are proteins.

In still other aspects, the biocompatible material of the kit is a biocompatible membrane, including, but not limited to amniotic membrane (including, but not limited to human amniotic membrane), SIS, fascia, dura matter, peritoneum, and pericardium.

In some aspects of the kit, the biocompatible material is in the shape of a tube.

In certain aspects, the second section of the kit of the invention is a border region. In certain aspects, particularly when the biocompatible material of the kit is in the shape of a tube, the border region can be at one or both ends of said material.

In yet other aspects, the kit also includes instructions for use of said tissue sealing device for the repair of a human tissue (including but not limited to human neural tissue).

In still another aspect, the invention encompasses a kit comprising an amniotic membrane conduit comprising a border region, and packaging materials therefor.

In certain aspects, particularly when the conduit of the kit of the invention is in the shape of a tube, the border region can be at one or both ends of said conduit.

In yet other aspects, the kit also includes instructions for use of said tissue sealing device for use of said conduit for peripheral nerve repair.

In yet another aspect, the invention provides a kit comprising a biocompatible membrane, a photosensitizer agent, and instructions for forming said biocompatible membrane into a tissue sealing device of the invention. In certain aspects, the kit also includes instructions for use of said tissue sealing device for the repair of a human tissue (including but not limited to human neural tissue).

In certain aspects, the photosensitizer agent of the kit is selected from the group consisting of xanthene (including, but not limited to Rose Bengal), flavin, phenothiazine, triphenylmethyl, cyanine, Mono azo dye, Azine mono azo dye, Phenothia-zine dye, rhodamine dye, Benzyphen-oxazine dye, oxazine, anthroqui-none dye, and porphyrin.

In still other aspects, the biocompatible material of the kit is a biocompatible membrane, including, but not limited to amniotic membrane (including, but not limited to human amniotic membrane), SIS, fascia, dura matter, peritoneum, and pericardium.

In some aspects of the kit, the biocompatible material is in the shape of a tube.

In certain aspects, the second section of the kit of the invention is a border region. In certain aspects, particularly when the biocompatible material of the kit is in the shape of a tube, the border region can be at one or both ends of said material.

Other aspects of the invention are described in the following disclosure, and are within the ambit of the invention.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of necessary fees.

The following Detailed Description, given by way of example, but not intended to limit the invention to specific embodiments aspects described, may be understood in conjunction with the accompanying drawings, which incorporated herein by reference. Various features and aspects of the present invention will now be described by way of non-limiting examples and with reference to the accompanying drawings, in which:

FIG. 1 shows (A) a human amniotic membrane conduit with the pink central area having been treated with 0.1% Rose Bengal and illuminated with a nd:YAG laser at 532 nm. The border region is shown as the not treated (i.e. not pink) terminal ends. (B) a collagen conduit with a free edge of the rolled collagen which has been sealed using PTB.

FIG. 2 shows conduits in situ. (A) Amnion conduit secured with sutures. Arrow shows the crosslinked central area which has maintained its tubular structure following rehydration. (B) Collagen conduit secured with sutures. Pink area indicates where the free edge has been treated with PTB. (C) Amnion conduit integrated with PTB. Arrow indicates where the proximal nerve end has been enveloped in the conduit. The conduit has been sealed to the nerve and itself using PTB. (D) Collagen conduit sealed with PTB.

FIG. 3a shows appearance of amnion conduits at twelve weeks post-operatively. (A) shows the nerve regeneration within an amnion conduit secured with sutures. (B) shows a PTB sealed conduit. The conduit is still present in both cases (arrows).

FIG. 3b shows gross appearance of conduits following harvest at 12 weeks post operatively. (A); amnion conduit secured with sutures. (B); amnion conduit sealed with PTB. The Rose Bengal stained conduit is still evident in both cases. (C); a thin band of neural tissue bridges the gap in the collagen conduit suture group. The conduit has been completely resorbed. (D); there was no neural regeneration in the collagen conduit PTB group. (E); autologous nerve graft.

FIG. 4 shows a chart showing (A) Gastrocnemius muscle mass preservation compared to the contralateral control muscle; and (B) Myocyte diameter preservation compared to contralateral control muscle. (NS=non significant. ** p<0.01)

FIG. 5 shows axonal regeneration within the conduits. (A) Autologous nerve graft showing organized regeneration with axons forming distinct fascicles. (B) Amnion nerve graft sealed with PTB. The area occupied by regenerating axons is large and there is minimal fibrous ingrowth. (C) Amnion nerve graft secure with sutures. The central area is occupied by axons but there is more fibrous tissue within the conduit. (Toluidine Blue 40x).

FIG. 6 shows 1 μμm sections from the midpoint of the nerve conduits which show regenerated axons in the (A) Autologous nerve graft, (B) Amnion conduit secured with sutures, (C) Amnion conduit secured with PTB and (D) Collagen conduit secured with sutures.

FIG. 7 shows 1 μμm sections from 5 mm distal to the nerve conduits show regenerated axons in the (A) Autologous nerve graft, (B) Amnion conduit secured with sutures, (C) Amnion conduit secured with PTB and (D) Collagen conduit secured with sutures. No regeneration is evident in the distal stump of nerves treated with collagen conduits sealed with PTB (E). (Toluidine Blue, original magnification 200.times.).

FIG. 8 shows a chart showing the total fiber counts measured within the conduit at the midpoint. NS=non significant. **p<0.01

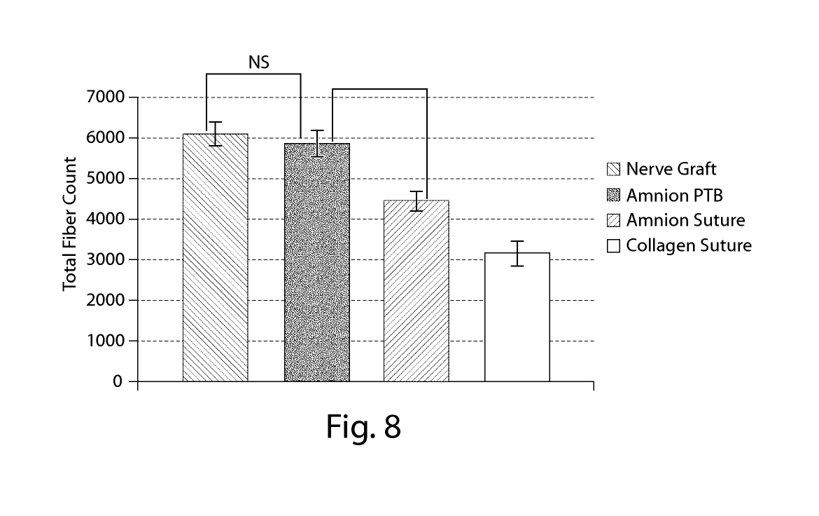

### DETAILED DESCRIPTION OF THE INVENTION

The present invention relates to a biocompatible membranes, tubes and conduits which comprising a photosensitizer which is capable of being crosslinked to form a three dimensional structure which can be implanted into a subject to assist in tissue bonding and nerve maintenance and development. Significantly, the membranes and other structures may be partially cross linked using a partial treatment with a photosensitizer thereby leaving one or more border regions which allows for further bonding of the structure to tissue or other biomaterial. This allows a generally rigid structure (formed by photo crosslinking) to be incorporated directly into tissues and act as conduits or other structures for healing and/or cell growth. This is particularly useful when a biological material or conduit is used to bridge between nerve ends.

### DEFINITIONS

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms may have the meanings ascribed to them below, unless specified otherwise. However, it should be understood that other meanings that are know or understood by those having ordinary skill in the art are also possible, and within the scope of the present invention.

As used herein, the term “biocompatible structure” refers to a structure having three-dimensions wherein the structure is compatible with living tissue or a living system. In that regard, a biocompatible structure is nontoxic and/or non-injurious to the living tissue or living system over the period of contact/exposure. Moreover, a biocompatible structure does not cause a substantial immunological reaction or rejection over the period of contact/exposure.

As used herein, the term “biocompatible material” refers to a material that includes molecules, such as protein molecules, that, when contacted with a photosensitizer agent and electromagnetic energy, will form cross-links between the proteins, and the photosensitizer agent. Biocompatible materials according to the invention include biological membrane and also biocompatible membranes composed of synthetic polymers such as, but not limited to, polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyesterspolyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, Teflon®, nylon silicon, and shape memory materials, such as poly(styrene-block-butadiene), polynorbornene, hydrogels, metallic alloys, and oligo(.epsilon.-caprolactone)diol as switching segment/oligo(p-dioxyanone)diol as physical crosslink. Other suitable polymers can be obtained by reference to The Polymer Handbook, 3rd edition (Wiley, N.Y., 1989).

By “biological membrane” or “biocompatible membrane” can mean, but in no way is limited to an organized layer or cells taken from any animal. In preferred embodiments, the biological membrane is an amniotic membrane. In other exemplary embodiments, the biological membrane can be taken from the amnion of a mammal, for example a cow, pig, sheep, or the like. In another preferred embodiment, the biological membrane may be taken from, for example, a human pregnancy, post partum. A biological membrane or biocompatible membrane can also include endothelium, fascia, pericardium, pleural lining, acellular muscle, blood vessel, dura matter, peritoneum, and mucosal membrane (such as small intestine submucosa, SIS). A biocompatible membrane can include synthetic membrane such as, but not limited to membranes made from an absorbable synthetic polymer, PGA, silicone, or other polymers such as polylactic acid (PLA), poly-L-lactic acid (PLLA), poly-D-lactic acid (PDLA), polyglycolide, polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyesterspolyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, Teflon®, nylon silicon, and shape memory materials, such as poly(styrene-block-butadiene), polynorbomene, hydrogels, metallic alloys, and oligo(.epsilon.-caprolactone)diol as switching segment/oligo(p-dioxyanone)diol as physical crosslink. It will be understood by those of skill in the art that one or more of the foregoing polymer constituents may be modified to include appropriate side chains (e.g., groups containing amino substituents) that permit cross-linking of the polymers.

As used herein, the term “shaped” with respect to, for example, a “shaped biocompatible material” refers to a predetermined physical or spatial form of a biocompatible material, biocompatible membrane, amniotic membrane, and the like. Shaped can refer to a material or membrane that is manipulated into a particular physical or spatial form such as a flat or substantially planar sheet, tube, conduit, sphere, or geometric solid (whether or not the shape has a hollow or solid interior). Shaped can also refer to a material having an intended three-dimensional physical or spatial form. Shaped can also refer to any of the foregoing physical and/or spatial configurations wherein the shaped structure is at least partially cross-linked so as to substantially retain the shape.

As used herein the term “preform” refers to a precursor to a shaped biocompatible material. A preform can refer to a biocompatible material that has not yet been set into a given shape. Alternatively, a preform can refer to a biocompatible material that has been set into a given shape, but which is not able to substantially retain that shape.

As used herein, the term “border region” refers to the portion of a biocompatible structure that forms a contact point with tissue of an individual into which the biocompatible structure has been implanted and to which the biocompatible structure is intended to be adhered; that is, the region of a biocompatible structure that will be cross-linked to the tissue of the individual into which it is implanted. For example, when the biocompatible structure is a tube or conduit, the border region is a region, present at one or both terminal ends of the tube or conduit, having at least 5% of the total length of the tube. Where the biocompatible structure has a three-dimensional shape other than a tube or conduit, the border region is at least a portion of the edge of the structure (such as, for example, the peripheral 1 mm or more of the biocompatible structure) that is intended to be adhered to the tissue of an individual into which it is implanted. The border region in such a structure can also be a portion of the biocompatible structure not at the edge, but which is nonetheless intended to be adhered to a tissue of the individual into which it is implanted. A border region also includes a region of a planar biocompatible membrane that, when the biocompatible membrane is shaped into a biocompatible structure, will form a border region of such biocompatible structure.

By “electromagnetic energy” can mean, but in no way limited to electromagnetic radiation, or the like. For example, electromagnetic radiation can include light having a wavelength in the visible range or portion of the electromagnetic spectrum, or in the ultra violet and infrared regions of the spectrum.

By “luminal anatomical structure” can mean, but in no way limited to a structure that is found on the luminal surface of, for example, a blood vessel or another anatomical conduit.

By “luminal surface” can mean, but in no way limited to the inner surface. A lumen is an interior space or cavity, for example, the interior of a blood vessel. The luminal surface of a blood vessel is the side facing the blood. For example, the luminal (or apical) side of an epithelial cell is the side that communicates with the lumen of the tube the epithelium lines.

The term “photosensitizer agent” can mean, but in no way limited to a chemical compound that produces a biological effect upon photoactivation or a biological precursor of a compound that produces a biological effect upon photoactivation, or the like. Exemplary photosensitizers can be those that absorb electromagnetic energy. The photosensitizers of the invention can include photosensitizer fragments and/or derivatives of known photosensitizers, which have the same or substantially the same function as the known photosensitizers, which means that function which is at least about 50% of the function of an original photosensitizer, more preferably about 60% or 70%, or still more preferably about 80% or 90%, or even more preferably about 95% or 99% the function of the known photosensitizer compound. A photosensitizer agent can be, but is not limited to a xanthenes, e.g., Rose Bengal and erythrosin; flavins, e.g., riboflavin; thiazines, e.g., methylene blue; porphyrins and expanded porphyrins, e.g., protoporphyrin I through protoporphyrin IX, coproporphyrins, uroporphyrins, mesoporphyrins, hematoporphyrins and sapphyrins; chlorophylis, e.g., bacteriochlorophyll A, phenothiazine, cyanine, Mono azo dye (e.g., Methyl Red), Azine mono azo dye (e.g., Janus Green B), Phenothia-zine dye (e.g., Toluidine Blue), rhodamine dye (e.g., Rhodamine B base), Benzyphen-oxazine dye (e.g., Nile Blue A, Nile Red), oxazine (e.g., Celestine Blue), and anthroqui-none dye (e.g., Remazol Brilliant Blue R). Exemplary photosensitizer agents may include, but are not limited to, Rose Bengal, riboflavin-5-phosphate, and methylene blue.

The photosensitizers of the invention can include “photoactive dyes,” which, as used herein, refers to those photosensitizers that produce a fluorescent signal when activated. The photoactive dyes of the invention may also be fragments and/or derivatives of a known photoactive dyes which have the same or substantially the same function as a known photoactive dye, which means a function that is at least about 50% of the function of a known photoactive dye, more preferably about 60% or 70%, or still more preferably about 80% or 90%, or even more preferably about 95% or 99% the function of a known photoactive dye.

Depending on the wavelength and power of light administered, a photosensitizer can be activated to fluoresce and, therefore, act as a photoactive dye, but not produce a phototoxic species. The wavelength and power of light can be adapted by methods known to those skilled in the art to bring about a phototoxic effect where desired.

By “photoactivatable membrane device” can mean, but in no way limited to a membrane that is capable of photoactivation, or the like. Photoactivation can be used to describe the process by which energy is absorbed by a compound, e.g., a photosensitizer, thus “exciting” the compound, which then becomes capable of converting the energy to another form of energy, preferably chemical energy.

The term “photosensitizer composition,” as used herein, refers to chemical constructs having one or more photosensitizers (or fragments and/or derivatives thereof), as well as other materials, such as linkers, backbones, targeting moieties and binders, that may be couple thereto.

As used herein, the term “fluorescent dye” refers to dyes that are fluorescent when illuminated with light but do not produce reactive species that are phototoxic.

Any compound or moiety of the invention that is fluorescent in one or more states can contain one or more “fluorophores,” which refers to a compound or portion thereof which exhibits fluorescence. The term “fluorogenic” refers to a compound or composition that becomes fluorescent or demonstrates a change in its fluorescence (such as an increase or decrease in fluorescence intensity or a change in its fluorescence spectrum) upon interacting with another substance, for example, upon binding to a biological compound or metal ion, upon reaction with another molecule or upon metabolism by an enzyme. Fluorophores may be substituted to alter their solubility, spectral properties and/or physical properties. Numerous fluorophores and fluorogenic compounds and compositions are known to those skilled in the art and include, but are not limited to, benzofurans, quinolines, quinazolines, quinazolinones, indoles, benzazoles, indodicarbocyanines, borapolyazaindacenes and xanthenes, with the latter including fluoresceins, rhodamines and rhodols as well as other fluorophores described in Haugland, Molecular Probes, Inc. Handbook of Fluorescent Probes and Research Chemicals, (9.sup.th ed., including the CD-ROM, September 2002), and include the photosensitizers, photoactive dyes, and fluorescent compounds and moieties of the invention.

As used herein, the term “detectable” or “directly detectable,” or the like, refers to the presence of a detectable signal generated from a compound of the invention, e.g., a photosensitizer, that is detectable by observation, instrumentation, or film without requiring chemical modifications or additional substances.

The term “subject” is used herein to refer to a living animal, including a human.

As used herein, the term “substantially retains” as it relates to a three-dimensional shape of a biocompatible structure refers to the retention of a three-dimensional shape to the extent that the biocompatible structure can be used for its intended purpose. “Substantially retains” refers to no greater than a 5% or more change in a given dimension of a biocompatible structure, for example no greater than a 5% change, 10%, 15%, 20%, 25%, 30%, 35%, 40%, 45%, 50%, 55%, 60%, 70%, 75% or 80% change in a given dimension, provided that the biocompatible structure can still be used for its intended purpose. For example, a linear human amniotic membrane tube intended for use as a conduit to permit nerve regeneration can undergo a 5% or more change in its linear shape (i.e., it can be curved), but only to the extent that it can function as a nerve conduit.

As used herein, the term “neural tissue” refers to neural tissue of the central or peripheral nervous system. Neural tissue can refer to peripheral nervous tissue, such as a peripheral nerve, a dorsal or ventral ramus, spinal nerve, or ganglion, and can also refer to central nervous tissue such as the spinal cord.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure.

### Biocompatible Materials

The present invention provides shaped biocompatible structures and tissue sealing devices that can be used for a wide array of applications such as nerve repair, surgical wound closure, stents, and the like. The structures described herein can be formed by contacting a biocompatible material with a photosensitizer agent, where upon application of electromagnetic energy, molecules in the material are able to form cross-links with the photosensitizer agent. The result is an increase in the rigidity of the biocompatible material such that the three-dimensional structure is formed and the structure substantially retains its desired shape. Biocompatible materials are materials that comprise molecules, such as protein molecules, that, when contacted with a photosensitizer agent and electromagnetic energy, will form cross-links between the cross-linkable molecules, and the photosensitizer agent. Biocompatible materials according to the invention can include biocompatible membranes, either natural or synthetic. Biocompatible membranes useful according to the invention can be biological membranes which are an organized layer or cells taken from an animal or produced synthetically. In one embodiment, the biological membrane is an amniotic membrane. In other exemplary embodiments, the biological membrane can be taken from the amnion of a mammal, for example a cow, pig, sheep, or the like. In another embodiment, the biological membrane may be taken from, for example, a human pregnancy, post partum. Biological membranes also include endothelium, fascia, pericardium, pleural lining, acellular muscle, blood vessel, dura matter, peritoneum, and mucosal membrane (such as small intestine submucosa, SIS). Biocompatible materials include biocompatible membranes composed of synthetic polymers such as, but not limited to, polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyesterspolyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, Teflon®, nylon silicon, and shape memory materials, such as poly(styrene-block-butadiene), polynorbomene, hydrogels, metallic alloys, and oligo(.epsilon.-caprolacto-ne)diol as switching segment/oligo(p-dioxyanone)diol as physical crosslink. Other suitable polymers can be obtained by reference to The Polymer Handbook, 3rd edition (Wiley, N.Y., 1989). One of skill in the art will readily appreciate that the foregoing polymers can be uses in biocompatible materials as described herein provided that they are adapted to be amenable to cross-linking by the methods of the invention (e.g., provided that the polymers contain suitable amino containing side chains or moieties).

### Amnionic Membranes for Forming Three-Dimensional Structures

The amniotic membrane is the translucent innermost layer of the three layers forming the fetal membranes, and is derived from the fetal ectoderm. The amniotic membrane contributes to homeostasis of the amniotic fluid. At maturity, the amniotic membrane is composed of epithelial cells on a basement membrane, which in turn is connected to a thin connective tissue membrane or mesenchymal layer by filamentous strands. In one embodiment of the invention, amniotic membrane is obtained from a human, although amniotic membrane may also be obtained from other mammals such as sheep, pig, cow.

Human amniotic membrane (HAM) is a substrate that can be photochemically modified to make shaped biocompatible structures. Native HAM is a transparent, 20 μm thick tissue that is flimsy in nature although somewhat tear resistant. Crosslinking of HAM provides enhanced rigidity and mechanical strength to the material

HAM in its native form can be used for photochemical tissue bonding to seal tissues by crosslinking at the interface between the HMA and the body tissue, e.g. peripheral nerve cornea, sclera and conjunctiva. In this process a photosensitizer agent is applied superficially to the HAM, which is then placed in intimate contact with the target tissue and illuminated in situ to form a tight seal or coverage of the native tissue, such as in sealing HAM nerve wraps.

The isolated amniotic membranes that can be used in the exemplary embodiment of the present invention may be obtained from a commercial source, for example from suppliers such as AmbioDry and AmbioDry2 from OKTO Ophtho and AMNIOGRAFT from Bio-Tissue. Alternatively, the amniotic membrane may be recombinant, or naturally occurring and sterilized. The amniotic tissue may be obtained postpartum and then preserved by any number of methods known to one of skill in the art (e.g. glycerol, lyophilization, gluteraldehyde, etc). Additionally, amniotic membranes that are derived from non-humans may be used. Methods for obtaining and preparing amniotic membrane are known in the art and are described, for example, in US20070031471, the contents of which are incorporated herein in their entirety.

The membranes of the exemplary embodiment of the present invention can be, for example, between 10 μm, 15 μm, 20 μm, 25 μm, 30 μm, 35 μm or more μm in thickness. In certain exemplary embodiments, the membrane is 20 μm in thickness, and is a human amniotic membrane.

### Photoactivation and Photosensitizer Agents

Photoactivation, as referred to herein, e.g., can be used to describe the process by which energy in the form of electromagnetic radiation is absorbed by a compound, e.g., a photosensitizer agent, thus “exciting” the compound, which then becomes capable of converting the energy to another form of energy, preferably chemical energy. The electromagnetic radiation can include energy, e.g., light, having a wavelength in the visible range or portion of the electromagnetic spectrum, or the ultra violet and infrared regions of the spectrum. The chemical energy can be in the form of a reactive species, e.g., a reactive oxygen species, e.g., a singlet oxygen, superoxide anion, hydroxyl radical, the excited state of the photosensitizer, photosensitizer free radical or substrate free radical species. The photoactivation process can involve an insubstantial transfer of the absorbed energy into heat energy. Preferably, photoactivation occurs with a rise in temperature of less than 3 degrees Celsius (C), more preferably a rise of less than 2 degrees C. and even more preferably, a rise in temperature of less than 1 degree C. as measured, e.g., by an imaging thermal camera that looks at the tissue during irradiation. The camera can be focused in the area of original dye deposit, e.g., the wound area, or on an area immediately adjacent the wound area, to which dye will diffuse. As used herein, a photosensitizer agent is a chemical compound that produces a biological effect upon photoactivation or a biological precursor of a compound that produces a biological effect upon photoactivation. Exemplary photosensitizers can be those that absorb electromagnetic energy, such as light. While not wishing to be bound by theory, the photosensitizer agent may act by producing an excited photosensitizer or derived species that interacts with tissue, e.g., amniotic membrane, to form a bond, e.g., a covalent bond or crosslink. Certain exemplary photosensitizers typically have chemical structures that include multiple conjugated rings that allow for light absorption and photoactivation. A number of photosensitizers are known to one of skill in the art, and generally include a variety of light-sensitive dyes and biological molecules. Examples of photosensitizer agent include, but are not limited to, xanthenes, e.g., Rose Bengal and erythrosin; flavins, e.g., riboflavin; thiazines, e.g., methylene blue; porphyrins and expanded porphyrins, e.g., protoporphyrin I through protoporphyrin IX, coproporphyrins, uroporphyrins, mesoporphyrins, hematoporphyrins and sapphyrins; chlorophylis, e.g., bacteriochlorophyll A, phenothiazine, cyanine, Mono azo dye (e.g., Methyl Red), Azine mono azo dye (e.g., Janus Green B), Phenothia-zine dye (e.g., Toluidine Blue), rhodamine dye (e.g., Rhodamine B base), Benzyphen-oxazine dye (e.g., Nile Blue A, Nile Red), oxazine (e.g., Celestine Blue), anthroqui-none dye (e.g., Remazol Brilliant Blue R), and photosensitive derivatives thereof. Exemplary photosensitizer agents according to the methods of the invention as described herein are compounds capable of causing a photochemical reaction capable of producing a reactive intermediate when exposed to light, and which do not release a substantial amount of heat energy. Some exemplary photosensitizers include Rose Bengal (RB); riboflavin-5-phosphate (R-5-P); methylene blue (MB); and N-hydroxypyridine-2-(1H)-thione (N-HTP).

In certain exemplary embodiments, a photosensitizer agent, e.g., RB, R-5-P, MB, or N-HTP, can be dissolved in a biocompatible buffer or solution, e.g., saline solution, and used at a concentration of from about 0.1 mM to 10 mM, preferably from about 0.5 mM to 5 mM, more preferably from about 1 mM to 3 mM.

A photosensitizer agent can be administered to a biocompatible material as described herein. Photosensitizer agents can be brushed or sprayed onto one or both surfaces of a biocompatible membrane prior to the application of electromagnetic energy. Other methods for applying photosensitizer agent (e.g., such as submerging the membrane in photosensitizer agent) can be envisioned by one of skill in the art. In one embodiment, photosensitizer agent is not applied to the entirety of the biocompatible membrane prior to forming a three-dimensional structure, and a portion of the biocompatible membrane is left free of photosensitizer agent. As described in further detail below, upon exposure to electromagnetic energy, the portion of the biological membrane that contains photosensitizer agent will form cross-links, while the portion that is free of photosensitizer agent will not form cross-links.

The electromagnetic radiation, e.g., light, can be applied to the tissue at an appropriate wavelength, energy, and duration, to cause the photosensitizer to undergo a reaction to affect the structure of the amino acids in the tissue, e.g., to cross-link a tissue protein, thereby creating a tissue seal. The wavelength of light can be chosen so that it corresponds to or encompasses the absorption of the photosensitizer, and reaches the area of the tissue that has been contacted with the photosensitizer, e.g., penetrates into the region where the photosensitizer is injected. The electromagnetic radiation, e.g., light, necessary to achieve photoactivation of the photosensitizer agent can have a wavelength from about 350 nm to about 800 nm, preferably from about 400 to 700 nm and can be within the visible, infra red or near ultra violet spectra. The energy can be delivered at an irradiance of about between 0.5 and 5 W/cm², preferably between about 1 and 3 W/cm². The duration of irradiation can be sufficient to allow cross-linking of one or more proteins of the tissue, e.g., of a tissue collagen. For example, in corneal tissue, the duration of irradiation can be from about 30 seconds to 30 minutes, preferably from about 1 to 5 minutes. The duration of irradiation can be substantially longer in a tissue where the light has to penetrate a scattering layer to reach the wound, e.g., skin or tendon. For example, the duration of irradiation to deliver the required dose to a skin or tendon wound can be at least between one minute and two hours, preferably between 30 minutes to one hour.

Suitable sources of electromagnetic energy can include but not limited to commercially available lasers, lamps, light emitting diodes, or other sources of electromagnetic radiation. Light radiation can be supplied in the form of a monochromatic laser beam, e.g., an argon laser beam or diode-pumped solid-state laser beam. Light can also be supplied to a non-external surface tissue through an optical fiber device, e.g., the light can be delivered by optical fibers threaded through a small gauge hypodermic needle or an arthroscope. Light can also be transmitted by percutaneous instrumentation using optical fibers or cannulated waveguides.

The choice of energy source can generally be made in conjunction with the choice of photosensitizer employed in the method. For example, an argon laser can be an energy source suitable for use with RB or R-5-P because these dyes are optimally excited at wavelengths corresponding to the wavelength of the radiation emitted by the argon laser. Other suitable combinations of lasers and photosensitizers are known to those of skill in the art. Tunable dye lasers can also be used with the methods described herein.

The photosensitizer agents of the current invention afford several beneficial aspects for cross-linking biocompatible membranes such as amnion. For example, the electromagnetic energy used to photoactivate the photosensitizer agent can typically penetrate further into tissues than other cross-linking energy sources, such as UV rays. Additionally, the current methods provide an alternative to using ionizing radiation to cross link the biocompatible membrane, which is well known to be detrimental to surrounding tissues. Furthermore, the photosensitizer agents useful in the invention can be non-toxic and the light initiation described herein provides a greater degree of control over the extent of cross-linking in the biocompatible membrane.

### Shaped Biocompatible Structures

The invention relates to shaped biocompatible structures (such as a tissue sealing device) that can be formed by placing a biocompatible material comprising a photosensitizer agent into a desired shape and exposing the membrane to electromagnetic energy, whereby cross-links are formed in the membrane, whereby the rigidity of the membrane is increased such that the membrane is able to substantially retain the desired shape. In one embodiment, the shaped biocompatible structure (i.e., tissue sealing device) comprises a first section of cross-linked moieties and a second section of noncross-linked moieties. The first section of cross-linked moieties confers rigidity to the structure. The second section of noncross-linked moieties is configured so that it is contactable with a tissue (e.g., nerve tissue) wherein the non-cross-linked moieties can be cross-linked with protein molecules of the tissue by contacting one or both of the structure and tissue with a photosensitizer agent and exposing the structure and tissue to electromagnetic energy. In one embodiment the noncross-linked section of a shaped biocompatible structure is a border region, meaning that it is a section that is intended to be used to bond the biocompatible structure to a host tissue. A border region can be located at any position on a biocompatible structure that is intended to be cross-linked to a host tissue.

Examples of biocompatible structures that can be formed using biocompatible membranes described herein include, but are not limited to, conduits, shunts, stents, patches, wound closure devices, and hernia repair patches. Biocompatible structures (i.e., shaped biocompatible structures) can also include scaffolding or framework structures on which additional tissues are grown or which can be implanted in the body to give three dimensional shape to tissue. Such framework structures include structures that mimic cartilaginous portions of the human body such as the ear or nose, or structures that are used in plastic surgical applications such as implants for the lips, cheeks, and the like. Three-dimensional biocompatible structures according to the invention can also be used to fill space in a body cavity or other body space to maintain the proper anatomical relationship of surrounding structures, such as, for example, inserting a shaped biocompatible structure into the body to fill the space previously occupied by an organ or other tissue.

Previous research has shown that the physical properties of a membrane, can be altered by photocrosslinking the constitutive proteins. For example, in one example, a tube was prepared by applying rose bengal to a strip of biological membrane, wrapping 3-4 layers around a rod, irradiating and then removing the rod [Irish Association of Plastic Surgeons, Galway, Ireland, May 10-12, 2007. Preparation and Integration of Nerve Conduits using a Photochemical Technique. O'Neill et al.]. Previous studies have also shown that flat layers of human amniotic membrane can be photocrosslinked together [unpublished].

Further, the amniotic membranes of the exemplary embodiment of the present invention may be modified to change their consistency. For example, amniotic membranes with enhanced rigidity as biocompatible devices are described in WO06002128.

A shaped biocompatible structure may be formed prior to deployment, during, or after deployment, in order to conform and/or alter the topology of the structure to which it is to be applied. In one embodiment, the shaped biocompatible structure is a tube that can be used as a conduit.

In one embodiment the shaped biocompatible structure is a conduit, such as a pre-formed conduit, made of partially cross-linked amniotic membrane. A piece of amniotic membrane is obtained (for example, as described hereinabove) and photosensitizer dye is partially applied to the central section of the membrane, leaving a portion of the membrane free of said photosensitizer agent (i.e., a border region). The membrane is then wrapped around a cylindrical support having an appropriate diameter and illuminated with electromagnetic energy, such as green light. Subsequent removal of the support results in a partially cross-linked amniotic membrane conduit for implantation. To implant the conduit, such as for peripheral nerve repair, photosensitizer agent is subsequently applied to the luminal surface of the border region, and the nerve stumps are inserted into the conduit and sealed by forming cross-links between the conduit and the peripheral nerve, for example, by applying electromagnetic energy in the form of green light.

In certain embodiments, the shaped biocompatible structure is designed to alter the topology of a luminal anatomic structure.

In one such example, the shaped biocompatible structure may be formed as a sheet of membrane, for example a sheet of amniotic membrane. This configuration may be preferable for use in imparting stability to one portion of the luminal anatomical structure.

In certain other examples, the intraluminal covering device that attaches to a luminal anatomic structure can at least partially cover the anatomical structure in a manner that either at least partially maintains the patency of said luminal anatomic structure.

In other examples, the membrane, preferably the exemplary biological membrane, attaches to a luminal anatomic structure that does not move within said structure following deployment. In other preferred examples, the biological membrane can attach to a luminal anatomic structure that at least partially covers the anatomical structure in a manner that either at least partially stabilizes of said luminal anatomic structure.

It may be preferred that the membrane attaches to a luminal anatomic structure does not damage said structure.

In another example, this topology may be used to repair a defect in an anatomical structure. In certain cases, it may be preferable to use the membrane of the invention to treat, repair, or cover only one portion of an anatomical structure, and leave the other portion of the anatomical structure intact. For example, to cover only a portion of the luminal anatomic structure that may utilize an alteration while leaving the remainder of the luminal anatomic structure intact. One example of this can be a covering or a stent, such as an intraluminal stent. Such a stent or covering can, for example, impart mechanical stability, act as a cover, or maintain at least partial patency of the structure it is covering (e.g. a luminal anatomic structure). The stent or covering may in certain examples be a resizable stent or covering that at least imparts mechanical stability, covers, or maintains at least partial patency of the anatomic structure. In this exemplary way, the stent or covering does not need to be fitted in diameter to be of a predetermined size, and overlapping areas of the shaped biocompatible structure take up the slack upon deployment of the device.

In another exemplary embodiments of the present invention, a number of different device patterns are described that enhance or enable different biological functions or capabilities.

The shaped biocompatible structure may be conformed to be in a certain exemplary geometry. For example, the shaped biocompatible structure may be conformed in a cylinder, a plane, a sphere, a geometry preformed to the contour of the tissue of interest, or preformed to a desired contour to effect the best clinical treatment. In certain preferred examples, the cylinder or tube is used as a conduit, stent or a covering.

In other examples, the edges of the shaped biocompatible structure are tapered, and in certain preferred embodiments, may contain projections. The projections can comprise amniotic membrane, metal struts, nitinol struts, plastic struts, or composites, such as Polytetrafluoroethylene (PTFE), teflon, plastic, rubber, nitinol, or biodegradable composites or the like.

The shaped biocompatible structure may be configured with holes. The shaped biocompatible structure may have be configured to have 1, 2, 3, 5, 10, 20, 50, 100, 150, 200, 300, 500, or more holes, or different number of holes. The holes in the membrane can be of any geometry and may be configured to allow for passage of intraluminal tissues such as, but not limited to, blood, bile, or lymph to pass through. The exemplary minimum diameter of the holes may be between 10, 20, 30 40, 50, 75, 100, 200, 400, 500, 600, 750 μm in order to allow the passage of red and white blood cells, but other diameters are conceivable, and are within the scope of the present invention. The exemplary pattern of holes may be configured to allow endothelial or epithelial cells or other cells to migrate through the shaped biocompatible structure.

The holes and intervening spaces may be configured to impart further mechanical stability to the shaped biocompatible structure. For example, the edges of the shaped biocompatible structure may be tapered to further significantly improve endothelial or epithelial cell migration.

Accordingly, it is one object of the present invention that the exemplary shaped biocompatible structure that attaches to a luminal anatomic structure promotes re-endothelialization or re-epithelialization of said anatomic structure. Such exemplary membrane device thereby can be configured to allow the endothelial or epithelial cells of the luminal anatomic structure to migrate and cover the biological membrane following deployment of the device. Promotion of this healing process can be facilitated by adjusting an exemplary biological membrane thickness, number and size of holes or openings, and by applying other pharmacological agents to the biological membrane device that facilitate said re-endo or re-epithelialization

The exemplary shaped biocompatible structure may be, in certain embodiments, comprised of layers of membrane, for example, amniotic membrane, configured to impart substantially more thickness and/or mechanical stability to the membrane device. The membranes of the exemplary embodiments of the present invention, may be modified to change shape or configuration. For example, the shaped biocompatible structures can be comprised of layers of one or more, for example, 2, 3, 5, 10, 20, 30, 50 or more membrane sheets. These sheets can be affixed to each other, in certain examples, by electromagnetic radiation.

In one exemplary embodiment, the layers may be affixed to one another by means of applying electromagnetic radiation to layers of amniotic membrane comprised of photoactivatable dye.

In the foregoing embodiments, a first section or portion of the biocompatible membrane is contacted with photosensitizer agent and a second section or portion is kept free of photosentisizing agent so as to create a noncross-linked border region in the final shaped structure. Shaped structures formed in this way will, therefore, only be partially cross-linked following application of electromagnetic energy. This partial cross-linking permits the shaped biocompatible structure to be deployed in a subject such that photosensitizer is applied to the non-cross linked border region of the structure (and/or is applied to the tissue to which the structure is to be adhered), wherein subsequent application of electromagnetic energy will function to create cross-links between the biocompatible membrane of the device at the border region and the target tissue to which the device is to be adhered. A border region may be formed at any location of the shaped structure that is intended for contact and bonding to the tissue of a subject. For example, the border region of a conduit may be located at either or both ends of the conduit, and/or may be located at some site in the conduit internal to the ends. In the context of a tube or conduit, a border region can occupy 5-40% of the total length of the tube or conduit. In one embodiment, as measured along the long axis of the tube or conduit, the border region can be 1 mm or more in length. For example the border region can be 2, 5, 10, 15, 20, 25, 30, 35, 40, 45, 50, 55, 60, 65, 70, 75, 80, 85, 90, 95, 100, 200, 300, 400, 500, 600, 700, 800, 900, or 1000 mm or more in length as measured along the long axis of the tube or catheter.

A border region need not be continuous with respect to the biocompatible membrane, but instead, may be discontinuous or located in discrete areas of the biocompatible membrane. For example, if the ultimate shape of a shaped biocompatible structure will have one or more specific points of contact with a host tissue, those points of contact can be created as border areas (by not applying a photosensitizer agent to the corresponding regions of the biocompatible membrane), regardless of whether the border region is at the edge of the biocompatible membrane, and regardless of whether the border region represents a continuous area of the biocompatible membrane.

A shaped biocompatible structure may be insertable or may be implantable. In one embodiment, the shaped structure may be pre-formed or partially pre-formed prior to implantation. The application of photosensitizer agent and/or electromagnetic energy may occur in situ in a subject or may be performed ex vivo prior to implantation of a device in a subject.

### Methods Using Shaped Biocompatible Structures

The shaped biocompatible structures (such as a tissue sealing device) described herein can be suitable for use in a variety of applications, including in vitro laboratory applications, ex vivo tissue treatments, but especially in in vivo procedures on living subjects, e.g., humans, and especially in nerve repair and repair of luminal anatomical structures.

In one embodiment, the shaped biocompatible structures described herein can be used as a tissue sealing device in nerve repair. A pre-formed conduit made from biocompatible membrane such as human amniotic membrane can be used to bridge a defect in neural tissue (such as a transection, nerve crush, partial transection, or other lesion), whereby an intraneural neurotrophic environment can be maintained within the conduit. In one embodiment, a biocompatible conduit as described herein can be used to bridge a gap between the cut ends of a peripheral nerve. It will be understood, however, that the phrase “bridge a gap” does not require a physical separation of the two ends of a nerve, but also includes a situation where the ends of a nerve are in contact with each other, but some or all of the nerve fibers have been severed or otherwise damaged. For example, a partially crosslinked conduit can be formed as described above. The site of nerve transection in a subject is then exposed under surgical conditions. Photosensitizer agent is then applied to the luminal surface of the conduit at least covering the border region, although photosensitizer may be applied a portion of the already cross-linked conduit. Photosensitizer agent may also or alternatively applied to the nerve that will be inserted into the conduit. Each cut end of the nerve is placed in the conduit and electromagnetic energy is applied to cross-link the border region of the conduit to the nerve stumps. In addition, one or more sutures may also be used to secure the ends of the transected nerve within the conduit. Sealing the nerve in the pre-formed conduit in this way preferably results in a watertight seal being formed between the neural tissue and the conduit. In addition to the foregoing, the conduit can be reinforced by placing one or more sutures through the conduit and tissue to be repaired. In one embodiment, the photosensitizer agent is only applied to one end of the conduit, while the other end of the conduit is secured with one or more sutures.

In a further embodiment a shaped biocompatible structure can be used in tissue repair applications such as hernia repair. For example, a piece of biocompatible membrane may be treated with photosensitizer agent, whereby a border region at the perimeter of the biocompatible membrane is left untreated:

### ##STR00001##

The membrane can then be exposed to electromagnetic energy whereby the treated portion of the membrane is cross-linked and has increased rigidity relative to the untreated border region. This partially cross-linked membrane patch can then be adhered to a facial, muscle, or other tissue layer in an individual having a hernia or other anatomical defect, wherein the border region is first treated with a photosensitizer agent, whereby subsequent exposure to electromagnetic energy bonds the membrane patch to the tissue of the individual by cross-linking the membrane at the border region with the tissue of the individual. In addition, the patch can be reinforced by placing one or more sutures through the border region and the tissue of the individual.

The invention also provides methods for stabilizing luminal anatomical structures and for treating or preventing atherosclerotic plaques.

The exemplary methods described herein can be used, for example, for tissue bonding. Tissue bonding can be used to seal anatomical sites, for instance, after injury, or after a surgical procedure, or as part of a prophylactic measure to prevent against a disease or pathological event. In one example, for instance, an exemplary biological membrane tissue bonding technique/procedure has been previously used to seal neurorrhaphy sites [Photochemical Sealing Improves Outcome Following Peripheral Neurorrhaphy. A. C. O'Neill, M. A. Randolph, K. E. Bujold, I. E. Kochevar, R. W. Redmond, J. M. Winograd submitted to Experimental Neurology], incorporated by reference in its entirety herein. In this example, Rose Bengal-stained biological membrane was wrapped around the repair site (rat sciatic nerve) and exposed to 30 J/cm2 (on each side) 532 nm (irradiance=0.5 W/cm2) using a frequency doubled Nd/YAG laser. For example, the biological membrane can additionally rapidly bond to vocal fold (epithelial, lamina propria and muscle layers) [unpublished], incorporated by reference in its entirety herein. In this example, bonding of a biological membrane to cornea (without epithelial layer) an energy density of 100 J/cm2 is typically used. Biological membrane has also been bonded to dermis, epidermis and tracheal submucosa.

Methods for stabilizing luminal structures can include the steps of contacting a biological membrane with a photosensitizer agent and deploying the biological membrane photosensitizer complex to the luminal anatomical structure of interest, and then applying electromagnetic energy, thereby adhering the biological membrane to the luminal anatomical structure. In one embodiment, the biological membrane is preformed into a shaped biocompatible structure such as a stent.

Another exemplary embodiment of the method according to the present invention can be provided for stabilizing a luminal anatomical structure. The exemplary method can comprise contacting a biological membrane with a photosensitizer agent and then deploying the biological membrane to the luminal anatomical structure in need of stabilization, applying electromagnetic energy to the biological membrane-photosensitizer complex in a manner effective to bond the tissue, and thereby stabilizing a luminal anatomical structure.

The invention also includes methods for treating or preventing an atherosclerotic plaque. The method comprises identifying an atherosclerotic plaque, contacting a biological membrane with a photosensitizer agent wherein a portion of the membrane is not contacted with the photosensitizer agent so as to form a border region, deploying the biological membrane to the atherosclerotic plaque, and applying electromagnetic energy to the biological membrane photosensitizer complex in a manner effective to bond the tissue, and thus treating or preventing an atherosclerotic plaque. In one embodiment, prior to deployment of the membrane, the membrane is exposed to electromagnetic energy to partially cross-link the membrane.

According to yet another embodiment of the present invention, methods for promoting one or more of cell growth and migration in a luminal anatomical structure of interest are provided. The exemplary method can comprise contacting a biological membrane with a photosensitizer agent, deploying the biological membrane photosensitizer complex to the luminal anatomical structure of interest, and applying electromagnetic energy, and thereby promoting cell growth and migration in a luminal anatomical structure of interest.

According to another embodiment of the invention, a shaped biocompatible structure can be formed and used to give structural shape to overlying tissues such as skin. For example, a shaped biocompatible structure can be used as an implant in cosmetic surgical applications, such as, for example, facial reconstruction (e.g, lip, cheek, brow or neck augmentation or reconstruction), scar repair, or repair of damage from traumatic injury that decreased the supporting structures underlying the skin or other tissue.

### Kits

In one embodiment, the invention provides kits comprising a shaped biocompatible structure as described herein and packaging materials therefor. In one embodiment, the kit includes a pre-formed shaped biocompatible structure (e.g., a tissue sealing device), while in another embodiment, the kit includes a Biocompatible material (e.g., a tissue sealing device pre-form) and a photo sensitizer agent with instructions for forming a shaped biocompatible structure. In either of the foregoing embodiments, the kit can also include written instructions that describe how to use the shaped biocompatible structure for a given purpose. For example, the instructions can describe how to use a tubular shaped biocompatible structure as a conduit for nerve repair. The instructions can include a description of methods for adhering a shaped biocompatible structure to anatomical structures such as nerve or other tissues, for stabilizing a luminal anatomical structure, for treating or preventing an atherosclerotic plaque, or for promoting one or more of cell growth and migration in or on a shaped biocompatible structure or tissue of interest as described herein. The exemplary kits can include packaging materials such as a container for storage, e.g., a light-protected and/or refrigerated container for storage of the shaped biocompatible structure and/or photosensitizer agent. A photosensitizer agent included in the kits can be provided in various forms, e.g., in powdered, lyophilized, crystal, or liquid form.

### Examples

This example is designed to show the difference between a human amniotic membrane of the invention as implanted by further photo cross-linking to indigenous nerve tissue as compared to the implantation of an amniotic membrane of the invention implanted by sutures and a collagen based membrane which is entirely cross-linked before implantation by sutures.

### Methods

### Preparation of Amnion Conduits

Human placenta was obtained with the approval of the institutional ethics committee. The placenta was washed with Earle's Balanced Salt Solution (Gibco, Grand Island, N.Y.) several times to remove any residual blood clots from the membrane. The amniotic membrane was peeled away from the chorion and placed on nitrocellulose paper (epithelial side down) which was cut into segments for storage. Segments were placed in storage medium which consisted of a 1:1 solution of 100% glycerol and Dulbeccos Modified Eagle's Medium (Gibco, Grand Island, N.Y.) with 1 ml of Penicillin-Streptomycin solution (Gibco, Grand Island, N.Y.) added to each 100 ml of the media. Segments were then frozen at −20.degree. C. overnight and −80.degree. C. for long-term storage. Segments were defrosted at room temperature immediately prior to conduit preparation.

2.times.3 cm segments of amnion were prepared and thoroughly rinsed in PBS for a period of 2 hours to remove all glycerol. Segments were laid out on a flat surface and blotted to remove excess fluid. 0.1% (w/v) Rose Bengal dye (Aldrich, Milwaukee, Wis.) in phosphate buffered saline was applied to the central 1 cm of the amnion segment on the epithelial surface and allowed to absorb for one minute. Excess dye was removed and the amnion was then wrapped around a 16 G angiocatheter to create the conduit tube.

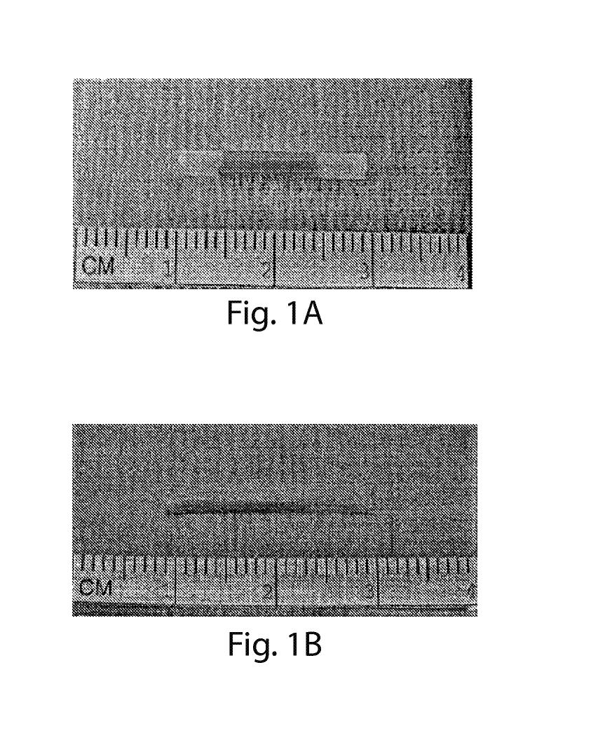

The dye treated area was exposed to green laser light at 532 nm from a Compass 415 continuous wave Nd/YAG laser (Coherent Inc., Santa Clara, Calif.), at an irradiance of .about.0.5 W/cm² for a period of 2 minutes. The angiocatheter was rotated during this time to ensure all areas were exposed to the laser. The amnion conduit was then dried on the angiocatheter at 60.degree. C. overnight (FIG. 1A).

### Preparation of the Collagen Conduit

A 1.times.2 cm segment of collagen sheeting (Collagen Matrix Film, Collagen Matrix Inc, NJ), was prepared and soaked in PBS. The collagen segment was then wrapped around a 16 G angiocatheter and allowed to dry for 30 minutes. Next, 0.1% (w/v) Rose Bengal solution was applied at the overlap and allowed to absorb for 1 minute before excess dye was removed The dye treated area was irradiated using the nd:YAG laser at an irradiance of 0.5 W/cm² for a period of 1 minute. Conduits were not further treated, as the material is partially cross-linked during manufacture. The collagen conduit was dried at room temperature overnight (FIG. 1B).

Both the amnion and collagen conduits were trimmed to 1.5 cm prior to use to permit a 2.5 mm overlap at each end and a 1 cm gap between the nerve ends.

### Surgical Procedure

The institutional Subcommittee on Research Animal Care at Massachusetts General Hospital approved all procedures in this study. Forty male Sprague Dawley rats (Charles River Laboratories, Wilmington, Mass.), weighing 250-350 g were anesthetized with an intraperitoneal injection of pentobarbital sodium (50 mg/kg, Abbott Laboratories Chicago, Ill.). The right sciatic nerve was then exposed through a dorso-lateral muscle splitting incision. Using an operating microscope (Codman, Randolph, Mass.), the nerve was dissected from the surrounding tissues and a 1 cm segment was sharply excised using a scalpel blade Animals were then randomized to one of six experimental groups:

Group 1: Autologous Nerve Graft (n=8)

The excised segment of nerve was reversed and replaced into the nerve gap. This served as an autologous nerve graft which is the current gold standard in the clinical management of nerve gaps. The reversed nerve graft was secured to the proximal and distal nerve stumps using 10/0 epineurial sutures (approximately 6 sutures at each end)

Group 2: Amnion Conduit (n=8)

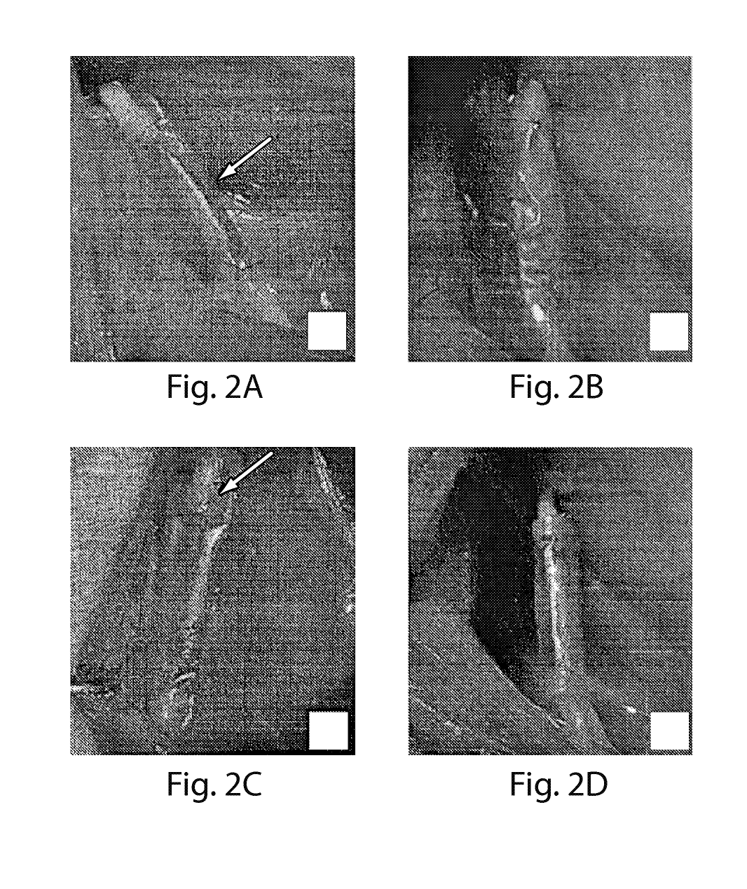

The proximal and distal segments of the severed nerve were inserted into the amnion conduit and secured with a single 10/0 nylon epineurial suture at either end (FIG. 2). The PTB treated area of the conduit maintained its tubular structure following rehydration and in-vivo placement.

Group 3: Amnion Conduit+PTB (n=8)

The proximal and distal segments of the severed nerve were inserted into the amnion conduit. The conduit/nerve overlap area was treated with 0.1% (w/v) Rose Bengal solution. The dye treated areas were irradiated using the nd:YAG laser at an irradiance of 0.5 W/cm² for a period of 1 minute at either end (FIG. 2).

Group 4: Collagen Conduit (n=8)

The proximal and distal segments of the severed nerve were inserted into the collagen conduit and secured with a single 10/0 nylon epineurial suture at either end (FIG. 2).

The proximal and distal segments of the severed nerve were inserted into the collagen conduit. The conduit/nerve overlap area was treated with dye and irradiated as described above (Group 3).

Following the above procedures the muscle and skin were closed using absorbable 4/0 polyglactin sutures (Ethicon, Somerville, N.J.). Animals were permitted to mobilize freely. They were housed in the animal facility of the Massachusetts General Hospital, where they had free access to water and rat chow.

### Evaluation

At 12 weeks post-operatively animals were re-anesthetized and the right sciatic nerve was exposed. The nerves were examined grossly for continuity, neuroma formation and evidence of nerve regeneration across the gap.

The nerve segment distal to the conduit was pinched with fine forceps and determined to have a positive pinch-reflex test if there was contraction of the leg muscles.

### Nerve Harvest and Histology

Conduits were harvested en bloc, including 5 mm of nerve proximally and distally and fixed in a 2% glutaraldehyde (Polysciences, Warrington Pa.)/2% paraformaldehyde (USB, Cleveland, Ohio) solution. Nerves were then post-fixed in 1% Osmium tetroxide, dehydrated in alcohol and embedded in araldite resin. 1 μm sections were made at the mid point of the conduit and immediately distal to the conduit using a microtome (Leica, Germany). Sections were stained with 0.5% (w/v) Toluidine blue for light microscopy. The total number of fibers present at the midpoint of the conduit and the 5 mm distal to the conduit were calculated from 200.times. images using Metamorph Imaging Software v4.6 (Universal Imaging Corporation™).

The mean fiber diameter and myelin thickness in the distal nerve were calculated for axons in one 200.times. field for each nerve.

### Gastrocnemius Muscle Preservation

The right gastrocnemius muscle and the contralateral normal gastrocnemius muscle were harvested from each animal and the wet weights recorded. The percentage of gastrocnemius muscle mass preserved was calculated (right gastrocnemius muscle mass/left gastrocnemius muscle mass.times.100) for each animal.

Muscles were then fixed in 4% paraformaldehyde for 24 hours prior to embedding in JB4 (Polysciences, Warrington Mass.). 2 μm sections were made and stained with Masons Trichrome for light microscopy. Myocyte diameters were measured using Metamorph Imaging Software v4.6 (Universal Imaging Corporation™).

### Statistics

Analysis of the data was performed using Sigmastat™ for Windows v2.3. Statistical significance was set at p-value<0.05. Analysis of Variance (ANOVA) and Tukeys pairwise comparison tests were used to evaluate the differences between the study groups.

### Results

### Gross Findings

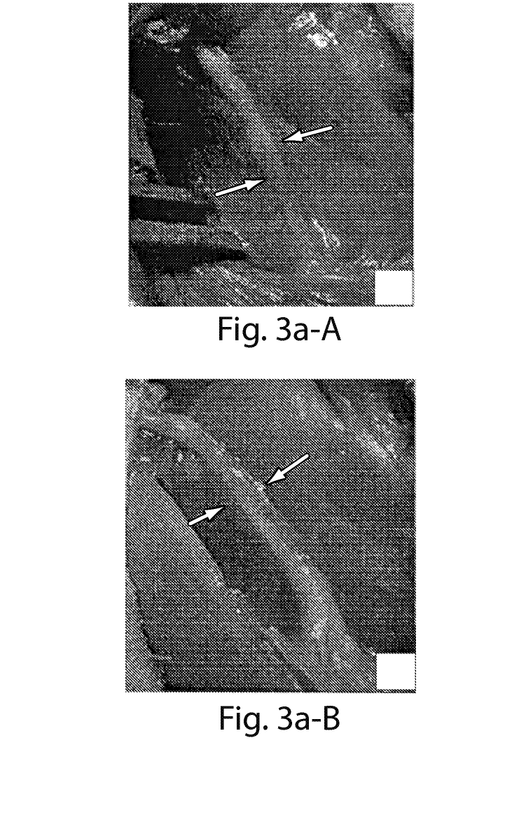

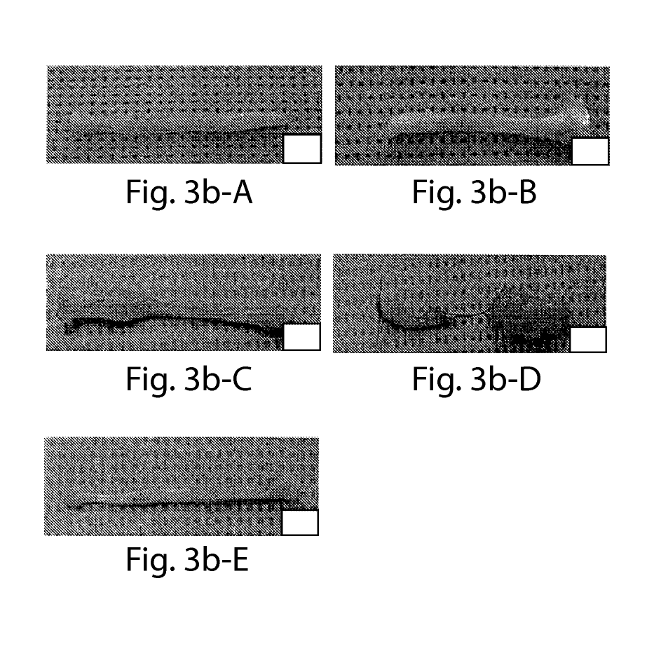

There was good regeneration across the autologous nerve grafts in all animals. The amnion conduits were still visible upon harvest at 12 weeks post-operatively. The Rose Bengal staining was apparent on the central section (FIG. 3b). The conduits could be seen to contain nerve tissue, crossing the entire length of the conduit (FIG. 3a). The collagen conduits had completely resorbed at 12 weeks. When collagen conduits were secured with sutures (group 4) a band of neural tissue connected the proximal and distal stumps in all cases (FIG. 3b). However, when collagen conduits were integrated using PTB (group 5) there was no neural regeneration across the gap (FIG. 3b). No further quantitative analysis was performed on nerve or muscles from this group. The pinch reflex was positive in all animals in all groups except the collagen conduit/PTB group.

### Muscle Mass

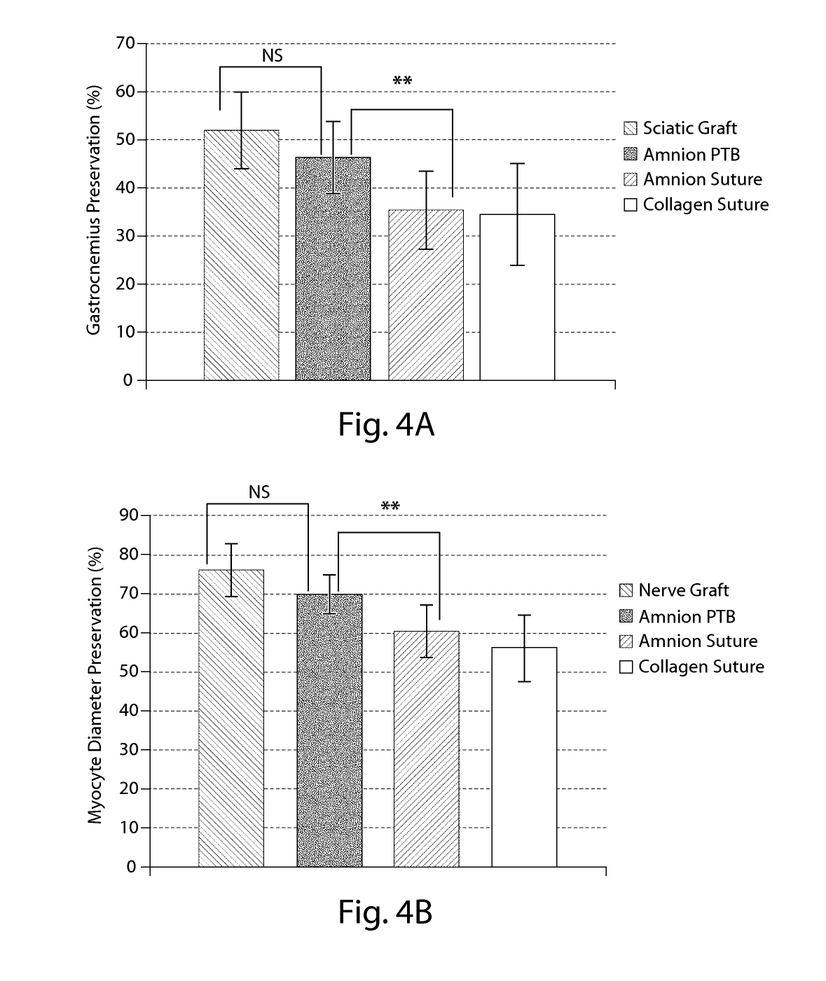

Gastrocnemius muscle mass preservation was greatest in the autologous nerve graft group (5 1.83+/−7.92) but this did not differ significantly from the amnion conduit/PTB group (46.07+/−7.56 p>0.05). When amnion conduits were secured with suture the muscle mass preservation was significantly lower than that seen in amnion conduit/PTB group (35.15+/−8.12 p<0.01). Lowest muscle mass preservation was observed in animals treated with collagen conduits (FIG. 4a).

### Muscle Histomorph

Gastrocnemius myocyte diameters were greatest in the autologous nerve graft group (76.25+/−6.36). The amnion conduit/PTB group showed significantly greater muscle fiber diameters than the animal treated with amnion conduits secured with sutures (69.85+/−4.69 vs 60.3+/−6.85 p<0.01).

### Nerve Histology

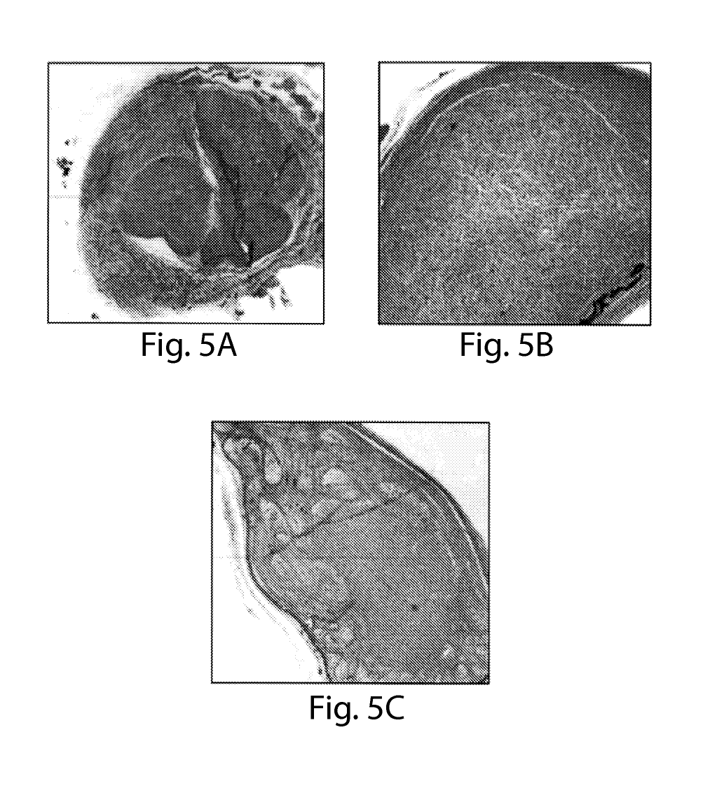

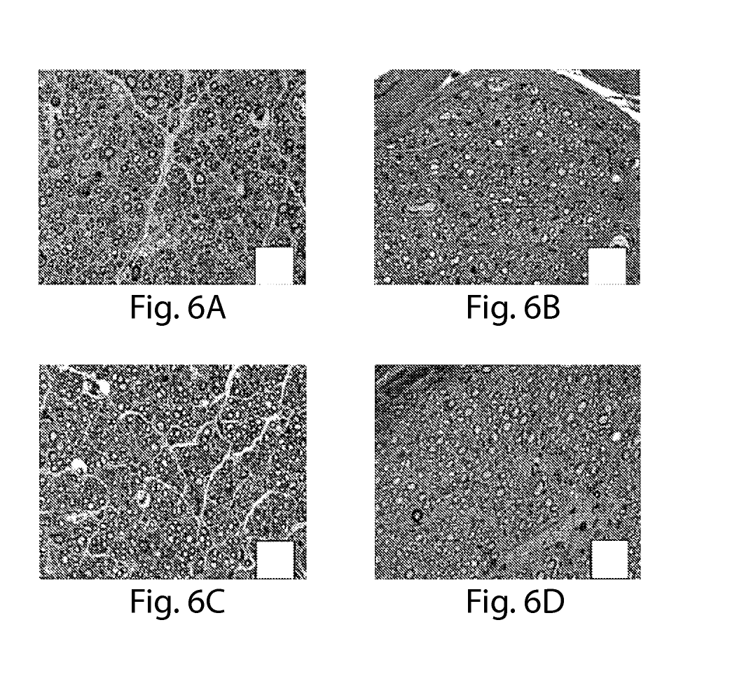

Myelinated fibers were present within the conduits in all cases in groups 1-4. Amnion conduits sealed with PTB contained significantly more myelinated fibers than amnion conduits secured with sutures (FIG. 5). In the amnion/PTB group nerve fibers filled the entire conduit while in the amnion/suture conduits fibers were concentrated in the center of the conduit with increased fibrous tissue peripherally (FIG. 5). Regeneration was best in the autologous nerve group but this was not significantly better than the amnion conduits sealed with PTB (FIGS. 5 and 6). Regeneration was also observed in the collagen conduits secured with sutures but the area of regeneration was reduced (FIG. 5).

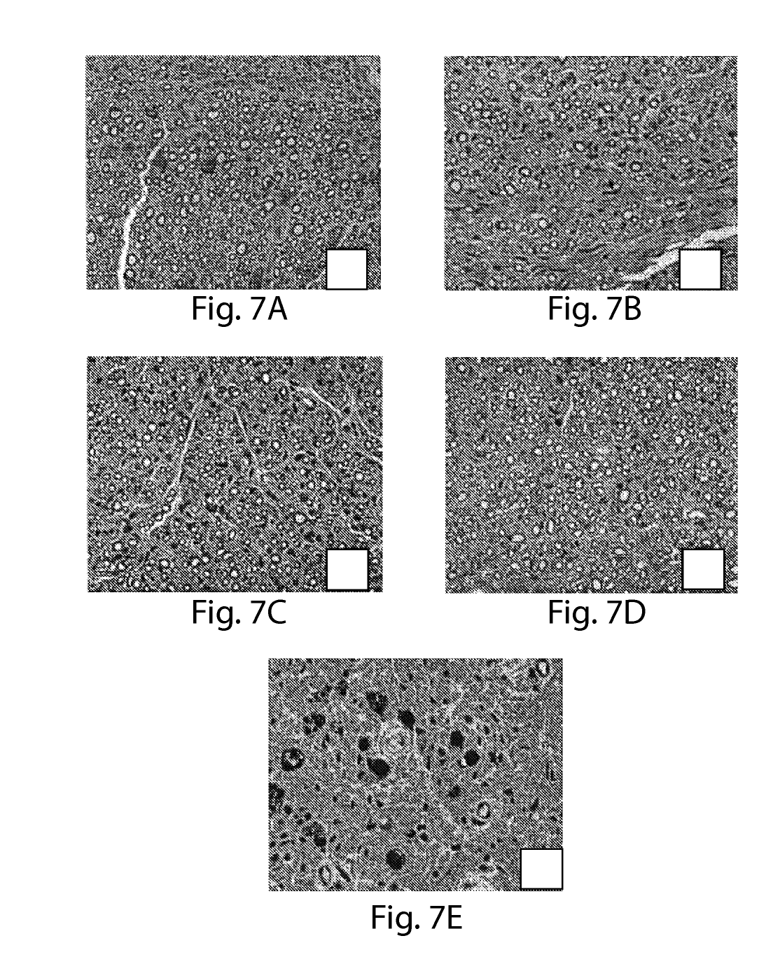

Myelinated fibers were also present distal to the conduits in all cases in groups 1-4 (FIG. 7). The total fiber counts followed the same pattern observed within the conduits, with the greatest number of fibers being present in the autologous nerve graft but this was not significantly superior to the amnion/PTB group. The amnion conduit sealed with PTB contained significantly more myelinated fibers in the distal nerve than the amnion/suture group. The lowest number of distal fibers was observed in the collagen suture group. Histology also confirmed the absence of regenerated fibers distally in the collagen/PTB group.

Fiber diameter and myelin thickness in the distal end of the amnion/PTB treated nerves were comparable to those observed in the autologous nerve group and significantly better than the amnion/suture group (Table 1).

TABLE-US-00001 TABLE 1 Sciatic Function Indices Group 4 Weeks 8 Weeks 12 Weeks Nerve Graft −92.4.+−. 3.8 −69.2.+−. 2.4*−60.3.+−. 3.2** Amnion/Suture −90.5.+−. 5.4 −76.8 .+−. 2.8 −71.8.+−. 2.9 Amnion/PTB −92.1.+−. 4.1 −72.9.+−. 3.2 −62.0.+−. 3.17** Sciatic function indices in each of the experimental groups at 4 week intervals post-operatively (*indicates statistical significance compared to all groups apart from the amnion/PTB group. **indicates statistical significance compared to other groups, p<0.01.).
TABLE-US-00002 TABLE 2 Nerve Histomorphometry Fiber Diameter. Myelin Thick. Group Fiber Count (μm) (μm) Nerve 5633.7.+−. 389.3** 4.62.+−. 1.41** 1.98.+−. 0.32** Graft Amnion/ 3578.5.+−. 386.7 2.05.+−. 1.54 0.98.+−. 0.36 Suture Amnion/ 5186.3.+−. 286.4** 4.11.+−. 1.67** 1.55.+−. 0.54** PTB Histomorphometric parameters five millimeters distal to the conduits at 12 weeks postoperatively (** indicates statistical significance, p<0.01). No regeneration occurred in the collagen/PTB group.

### EQUIVALENTS

Those skilled in the art will recognize, or be able to ascertain using no more than routine experimentation, many equivalents of the specific embodiments of the invention described herein. Such equivalents are intended to be encompassed by the following claims.

What is claimed is:

1. A method of forming a shaped tissue sealing device, said method comprising: contacting at least a first section of a biocompatible material with a photosensitizer agent, wherein at least a second section of said biocompatible membrane is not contacted with said photosensitizer agent; forming said biocompatible material into a desired shape; applying electromagnetic energy to said biocompatible material in an amount and duration sufficient to form cross-links between moieties of said first section, whereby a shaped tissue sealing device is formed.

2. The method of claim 1, wherein said biocompatible material is selected from the group consisting of amniotic membrane, SIS, fascia, dura matter, peritoneum, and pericardium.

3. The method of claim 2, wherein said biocompatible material is amniotic membrane.

4. The method of claim 1, wherein said second section is a border region.

5. The method of claim 1, wherein said shaped tissue sealing device has a three-dimensional shape.

6. The method of claim 5, wherein said three-dimensional shape is a tube.

7. The method of claim 1, wherein said photosensitizer agent is selected from the group consisting of xanthene, flavin, phenothiazine, triphenylmethyl, cyanine, Mono azo dye, Azine mono azo dye, Phenothia-zine dye, rhodamine dye, Benzyphen-oxazine dye, oxazine, anthroqui-none dye, and porphyrin.

8. The method of claim 7, wherein said xanthene is Rose Bengal.

9. The method of claim 1, wherein the electromagnetic energy is applied at an irradiance less than 1.5 W/cm².

10. The method of claim 1, wherein the electromagnetic energy is applied at an irradiance of about 0.50 W/cm².

11. The method of claim 1, wherein said electromagnetic energy is not applied to said second section.

12. The method of claim 1, further comprising the step of obtaining said cross-linkable material.

13. The method of claim 1, wherein said moieties are proteins.

14. The method of claim 4, wherein said biocompatible material is in the shape of a tube, and said border region is located at an end of said tube.

15. The method of claim 14, wherein said border region is at both ends of said tube.

16. The method of claim 3, wherein said biocompatible material is human amniotic membrane.
